(19) United States
(12) Patent Application Publication
(10) Pub. No.: US 2020/0359900 A1
(43) Pub. Date: Nov. 19, 2020

(54) BIOMETRIC SIGNAL MEASURING DEVICE

(71) Applicant: SAMSUNG ELECTRONICS CO., LTD., Suwon-si (KR)

(72) Inventors: Long Yan, Hwaseong-si (KR); Hyunsurk Ryu, Hwaseong-si (KR); Seoungjae Yoo, Seongnam-si (KR)

(73) Assignee: SAMSUNG ELECTRONICS CO., LTD., Suwon-si (KR)

(21) Appl. No.: 16/739,721

(22) Filed: Jan. 10, 2020

(30) Foreign Application Priority Data
May 17, 2019 (KR) 1020190057878

(51) Int. Cl. A61B 5/00

(52) U.S. Cl. CPC A61B 5/0062

(57) ABSTRACT

A biometric signal measuring device includes a light source configured to output a light signal to an object; a pixel array connected to a plurality of row lines and a plurality of column lines, and comprising a plurality of unit pixels configured to sense a reflected light signal corresponding to a reflection of the output light signal by the object; a binning controller configured to group the plurality of unit pixels to obtain one or more macro pixels; and a controller configured to drive the light source and to obtain a biometric signal of the object, based on a pixel signal generated by each of the one or more macro pixels according to the reflected light signal.

### CROSS-REFERENCE TO RELATED APPLICATION(S)

This application claims priority to Korean Patent Application No. 10-2019-0057878, filed on May 17, 2019 in the Korean Intellectual Property Office, the disclosure of which is incorporated herein by reference in its entirety.

### BACKGROUND

### 1. Field

Apparatuses and methods consistent with one or more exemplary embodiments relate to a biometric signal measuring device.

### 2. Description of Related Art

Recently, there has been growing demand for electronic devices having healthcare functions. Thus, a biometric signal measuring device capable of measuring various biometric signals, such as a user's heart rate and blood oxygen saturation (SpO2), has been gaining attention. A biometric signal measuring device is desired to have a significantly reduced size and power consumption as an electronic device. Accordingly, research has been actively conducted on an apparatus and a method for measuring a biological signal using an image sensor.

### SUMMARY

Aspects of one or more exemplary embodiments provide a biometric signal measuring device in which miniaturization thereof may be implemented using a pixel array of an image sensor and a user's biometric signals may be measured with relatively low power.

According to an aspect of an exemplary embodiment, a biometric signal measuring device includes: a light source configured to output a light signal to an object; a pixel array connected to a plurality of row lines and a plurality of column lines, and including a plurality of unit pixels configured to sense a reflected light signal corresponding to a reflection of the output light signal by the object; a binning controller configured to group the plurality of unit pixels to obtain one or more macro pixels; and a controller configured to drive the light source and to obtain a biometric signal of the object, based on a pixel signal generated by each of the one or more macro pixels according to the reflected light signal.

According to an aspect of an exemplary embodiment, a biometric signal measuring device includes: a pixel array including a plurality of unit pixels, the plurality of unit pixels being grouped into one or more macro pixels; a readout circuit configured to detect, during a first time period, a pixel signal output by each of the one or more macro pixels based on an external light signal, and to convert the detected pixel signal into a digital signal during a second time period; and a control logic configured to control operation timings of the pixel array and the readout circuit, and to sum the digital signal to generate a biometric signal.

According to an aspect of an exemplary embodiment, a biometric signal measuring device includes: a plurality of unit pixels connected to a plurality of row lines and a plurality of column lines; a binning controller configured to group the plurality of unit pixels to obtain one or more macro pixels; and a controller configured to convert a pixel signal generated by each of the one or more macro pixels based on an external light signal into a digital signal, and to generate a biometric signal by applying a predetermined weight to the digital signal.

According to an aspect of an exemplary embodiment, a biometric signal measuring method using a biometric signal measuring device, includes: controlling to output a light single from a light source of the biometric signal measuring device to an object; controlling to sense, by a plurality of unit pixels in a pixel array, a reflected light signal corresponding to a reflection of the output light signal by the object; and obtaining a biometric signal of the object based on a pixel signal generated by each of one or more macro pixels according to the reflected light signal, the one or more macro pixels each including unit pixels grouped together from among the plurality of unit pixels.

### BRIEF DESCRIPTION OF DRAWINGS

The above and other aspects, features, and advantages of certain exemplary embodiments will be more clearly understood from the following detailed description, taken in conjunction with the accompanying drawings, in which:

FIG. 1 is a view of a biometric signal measuring device according to an exemplary embodiment;

FIG. 2 is a simplified view of a sensing device according to an exemplary embodiment;

FIG. 3 is a diagram of a pixel array that may be included in the sensing device of FIG. 2;

FIGS. 4A and 4B are diagrams showing the structure of a unit pixel that may be applied to one or more exemplary embodiments;

FIGS. 5A to 5C are diagrams showing a vertical structure of a unit pixel that may be applied to one or more exemplary embodiments;

FIGS. 6A to 6C, 7A and 7B are diagrams for explaining a method of constructing a macro pixel according to one or more exemplary embodiments;

FIGS. 8A and 8B are block diagrams showing a configuration of a readout circuit that may be included in the sensing device of FIG. 2;

FIGS. 9 to 11 are views for explaining the operation of the biometric signal measuring device according to an exemplary embodiment;

FIG. 12 is a block diagram schematically showing an apparatus for measuring a biometric signal according to an exemplary embodiment; and

FIGS. 13 to 15 are views illustrating an example of an electronic apparatus including a biometric signal measurement apparatus according to one or more exemplary embodiments.

### DETAILED DESCRIPTION

Hereinafter, exemplary embodiments will be described in detail with reference to the accompanying drawings. The same reference numerals are used for the same constituent elements in the drawings, and redundant description of the same constituent elements will be omitted.

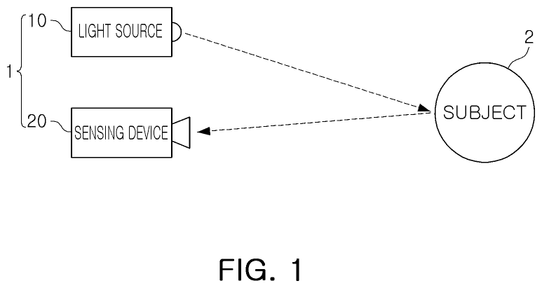

FIG. 1 is a view of a biometric signal measuring device 1 according to an exemplary embodiment.

Referring to FIG. 1, an apparatus 1 for measuring a biometric signal (e.g., biometric signal measuring device 1) according to an exemplary embodiment may include a light source 10 and a sensing device 20. When the biometric signal measurement device 1 receives a biometric signal measurement request from a user, the biometric signal measurement device 1 may output a light signal by turning on the light source 10.

The light source 10 may include at least one light emitting element. For example, the light source 10 may include at least one of a light emitting diode (LED), a laser diode, a vertical cavity surface emitting laser (VCSEL), and a phosphor. A plurality of light emitting elements included in the light source 10 may be arranged in an array form.

The sensing device 20 may detect a light signal scattered or reflected by living tissue of the object 2 to generate a living body signal. The sensing device 20 may include a pixel array that generates an electric signal in response to the detected light signal, and a controller that generates a living body signal using the electric signal generated by the pixel array. In an exemplary embodiment, the biometric signals may include a pulse wave signal (PPG), an electrocardiography (ECG) signal and an electromyography (EMG) signal. The object 2 may be a body part of the user who touches or is adjacent to the sensing device 20 and a body part that facilitates generation of a biometric signal. For example, in the case of measuring the pulse wave signal PPG, the object 2 may be a wrist, an ear, or the like, which is a body part relatively thin in skin tissue and high in blood vessel density.

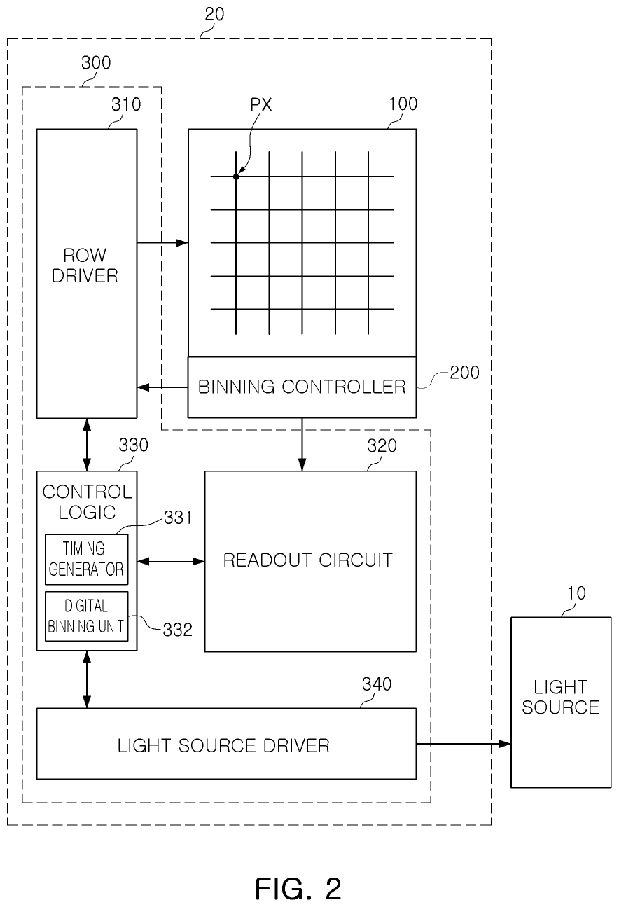

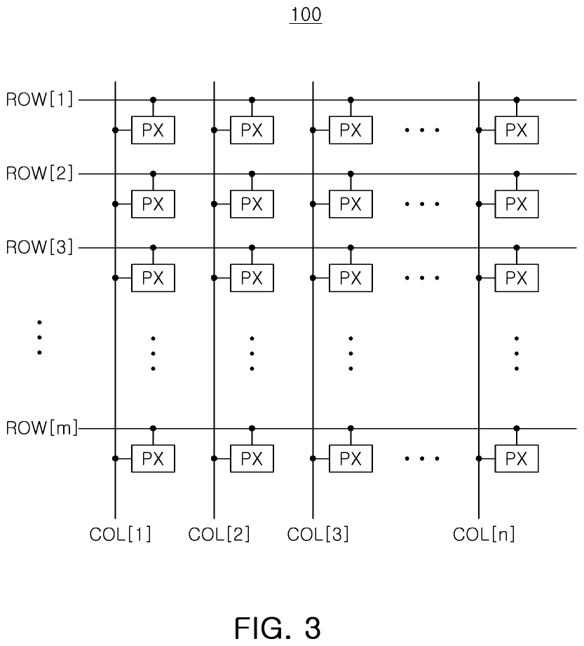

FIG. 2 is a simplified illustration of a sensing device 20 in accordance with an exemplary embodiment, and FIG. 3 is a view of a pixel array 100 that may be included in the sensing device 20 of FIG. 2.

Referring first to FIG. 2, the sensing device 20 may include a pixel array 100, a binning controller 200, and a controller 300.

The pixel array 100 may include a plurality of unit pixels PX. When a plurality of unit pixels PX are arranged in a matrix form, the unit pixels PX may be disposed at the intersections of the plurality of row lines and the plurality of column lines. An example of a pixel array 100 that may be applied to one or more exemplary embodiments is illustrated in FIG. 3.

Referring to FIG. 3, a plurality of unit pixels PX arranged in an array form may be connected to a plurality of row lines ROW[1] to ROW[m] and a plurality of column lines COL[1] to COL[n]. In an exemplary embodiment, the unit pixels PX may include a pixel responsive to a light signal of a particular wavelength suitable for acquiring a biometric signal. For example, the unit pixels PX may include pixels that respond to near infrared rays reflected by skin tissue or blood vessels.

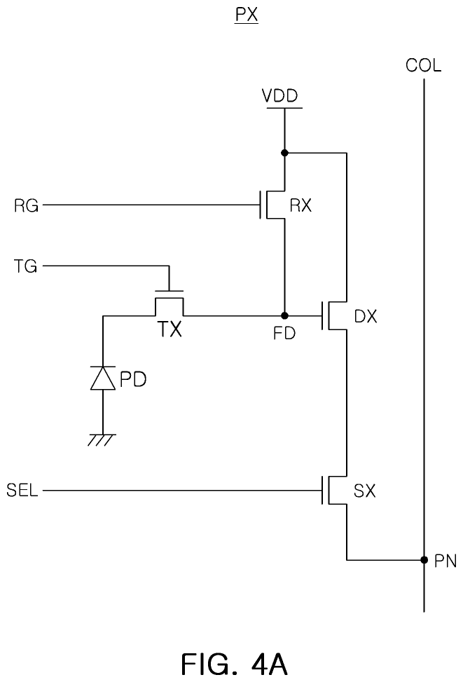

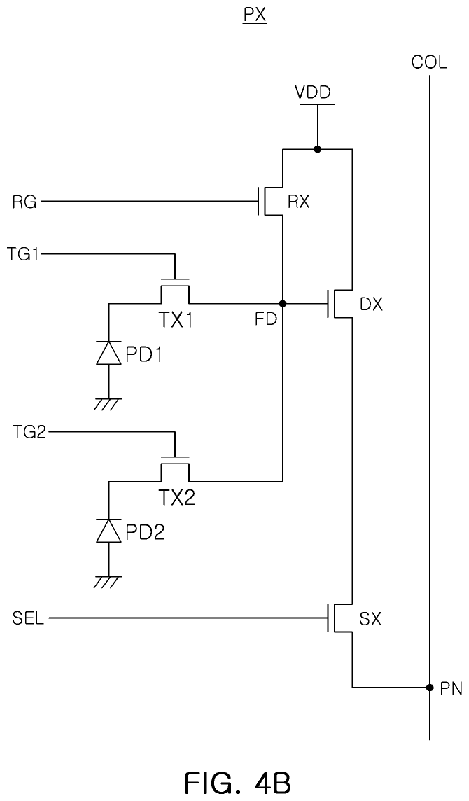

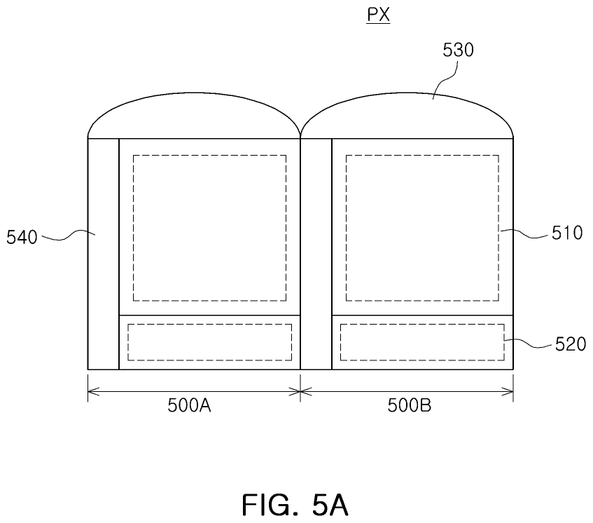

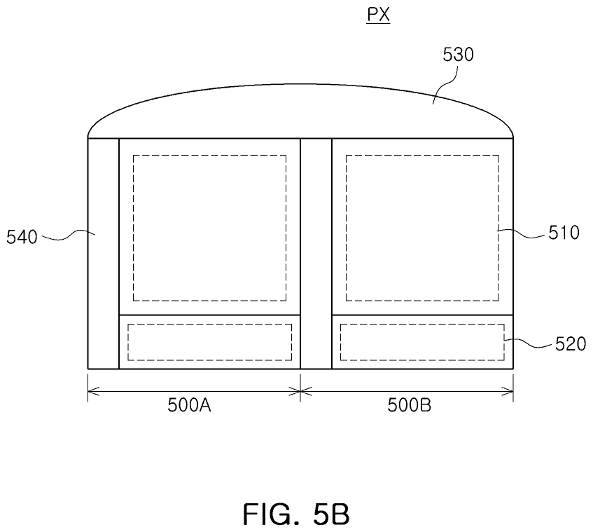

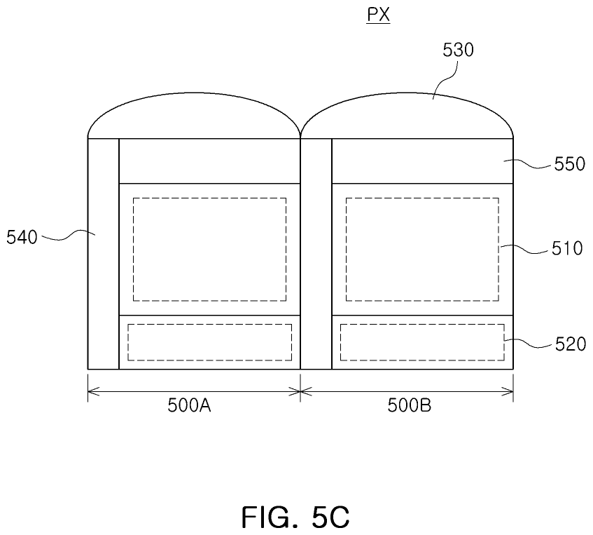

Specific examples of the unit pixel PX are as shown in FIGS. 4A, 4B, and 5A to 5C.

FIG. 4A shows a unit pixel PX having a 4T structure according to an exemplary embodiment, and FIG. 4B shows a unit pixel PX having a structure in which two photodiodes PD1 and PD2 share one floating diffusion FD according to an exemplary embodiment.

Referring to FIG. 4A, each unit pixel PX may include a photodiode PD and a pixel circuit. The pixel circuit may include a floating diffusion FD, a reset transistor RX, a driving transistor DX, a selection transistor SX, and a transfer transistor TX.

When the light signal output from the light source is reflected from the object and is incident on the pixel array 100, the photodiode PD may generate charges in response to the incident light. The charge generated by the photodiode PD may be accumulated in the floating diffusion FD.

When the reset transistor RX is turned on by the reset control signal RG, the voltage of the floating diffusion FD may be reset to the power supply voltage VDD. When the voltage of the floating diffusion FD is reset, the selection transistor SX is turned on by the selection control signal SEL so that the reset voltage may be output to the column line COL through the pixel node PN.

If the transfer transistor TX is turned on by the transfer control signal TG after the reset voltage is output to the column line COL, the charge generated by the photodiode PD may be transferred to the floating diffusion FD.

The driving transistor DX may operate as a source-follower amplifier that amplifies the voltage of the floating diffusion FD. When the selection transistor SX is turned on by the selection control signal SEL, the pixel voltage corresponding to the amount of charge generated by the photodiode PD may be output to the column line COL through the pixel node PN.

Referring to FIG. 4B, each unit pixel PX according to an exemplary embodiment may include a first photodiode PD1, a second photodiode PD2, and a pixel circuit. The pixel circuit may include a floating diffusion FD, a reset transistor RX, a driving transistor DX, a selection transistor SX, a first transfer transistor TX1, and a second transfer transistors TX2.

The first photodiode PD1 and the second photodiode PD2 may share a floating diffusion FD and a reset transistor RX. The charge generated by the first photodiode PD1 and the second photodiode PD2 may be accumulated in the floating diffusion FD. When the selection transistor SX is turned on by the selection control signal SEL, the pixel voltage (pixel signal) corresponding to the total amount of charges generated by the first photodiode PD1 and the second photodiode PD2 is supplied to the pixel node PN and the column line COL.

FIGS. 5A and 5B show a vertical structure of a unit pixel 500A and 500B that may be applied to one or more exemplary embodiments.

Referring to FIG. 5A, each unit pixel 500A and 500B may include a photoelectric element 510, a pixel circuit 520, and a microlens 530. The unit pixels 500A and 500B may be arranged in the form of an m×n matrix (where m and n are natural numbers) in the pixel array, and a separation for preventing crosstalk may be provided between the adjacent unit pixels 500A and 500B. An isolation region 540 may be formed or provided. In some examples, the isolation region 540 may be formed with deep trench isolation (DTI).

The photoelectric element 510 (or optoelectronic device) may be disposed below the microlens 530 and the pixel circuit 520 may be disposed below the photoelectric element 510. In some exemplary embodiments, the unit pixels PX may share a microlens in a predetermined unit. For example, referring to FIG. 5B, two adjacent unit pixels 500A and 500B may share a single microlens 530. However, it is understood that this is just an example, and other exemplary embodiments are not limited thereto. For example, n adjacent unit pixels PX may share one microlens, where n is an integer greater than or equal to 2. In this case, the n unit pixels PX may be grouped into various shapes such as a line or a rectangle.

Referring to FIG. 5C, each unit pixel 500A and 500B may further include an optical filter 550. In an exemplary embodiment, the optical filter 550 may include a color filter that only passes light of a particular color. In addition, the optical filter 550 may include an IR band pass optical filter that allows only light in the near infrared band reflected by skin tissues or blood vessels to pass therethrough.

Referring back to FIG. 2, the binning controller 200 may form at least one macro pixel (MP) by grouping a plurality of unit pixels PX included in the pixel array 100. In an exemplary embodiment, the binning controller 200 may configure the macro pixel MP by performing a binning operation on at least some of the row lines and/or at least some of the column lines. In this case, the readout operation for the macro pixel MP may be performed simultaneously on the binned row lines and/or the binned column lines.

The binning controller 200 may also constitute a macro pixel MP by using a unit pixel PX sharing a floating diffusion FD as shown in FIG. 4B.

The biometric signal measuring device 1 may reduce the number of unit pixels PX read out from the pixel array 100 by constructing the macro pixel MP to reduce the readout time and the number of analog-to-digital converters.

In addition, the biometric signal measurement apparatus 1 may perform spatial oversampling by constructing the macro pixel MP, and may increase the resolution of the output voltage through the pixel node of the macro pixel MP. For example, when a macro pixel MP having a size of 2×1 including two unit pixels PX is constructed, the resolution of the output voltage through the pixel node of the macro pixel MP may be increased by 0.5 bits. When a macro pixel (MP) having a size of 128×128 is configured for the pixel array having a dynamic range (DR) of 12-bits, the dynamic range DR of the pixel array is set to 19.

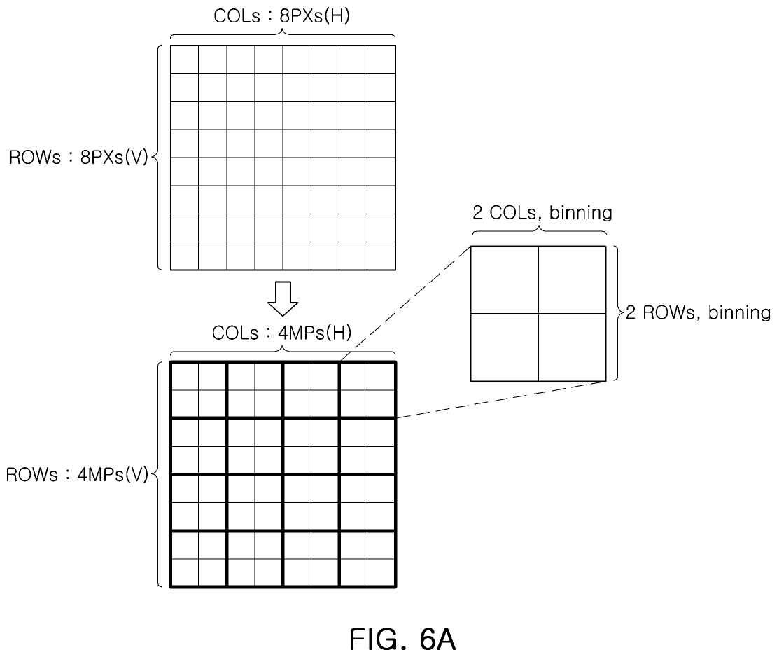

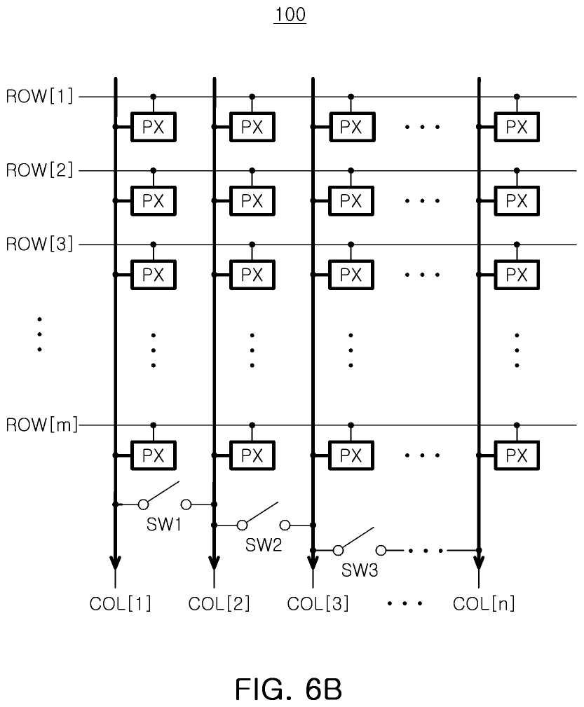

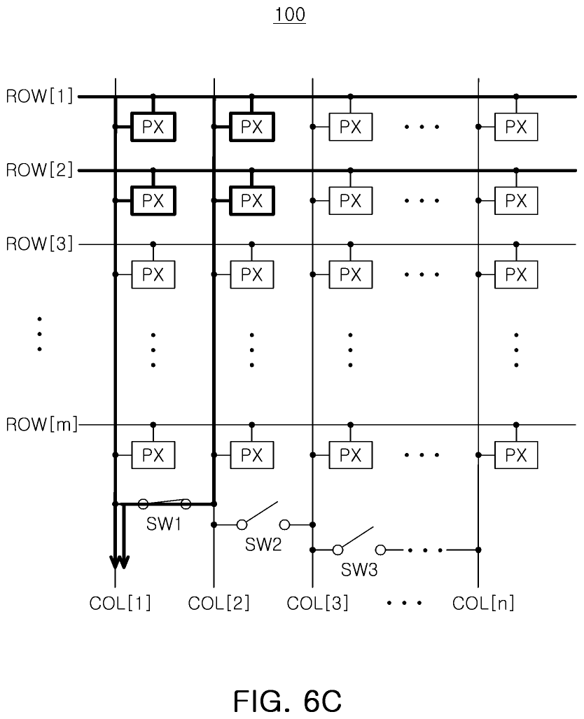

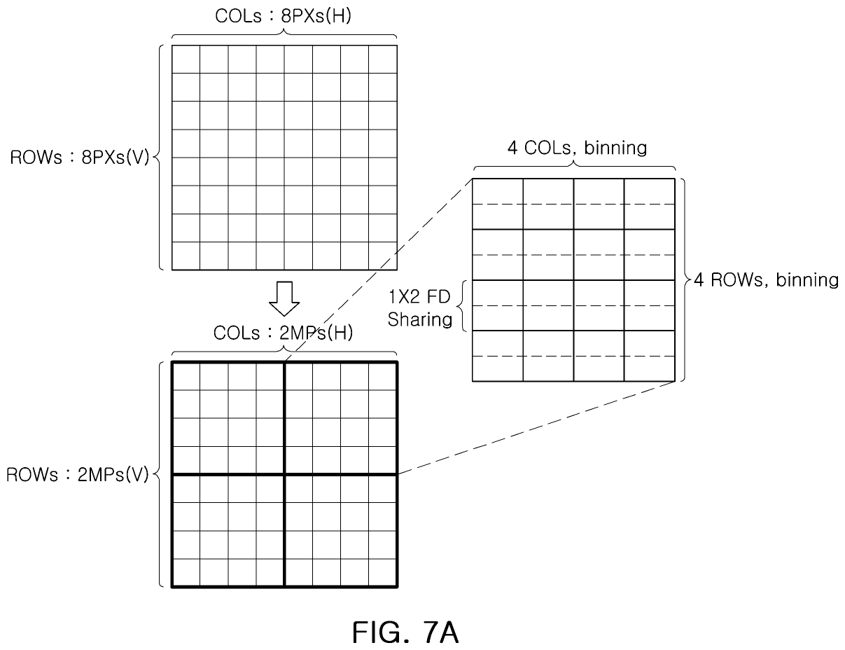

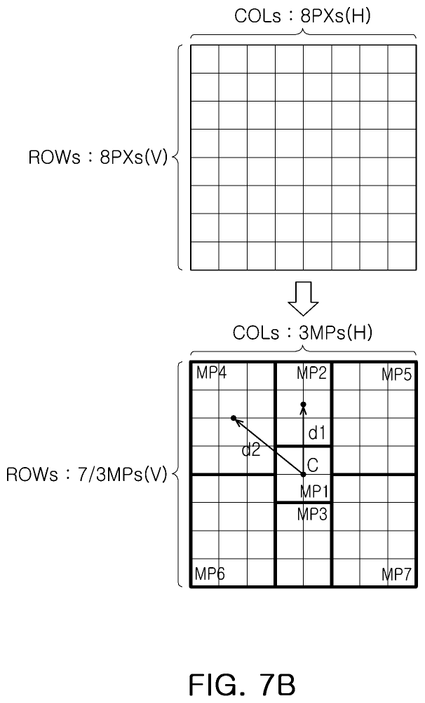

FIGS. 6A to 6C, 7A, and 7B are diagrams for explaining a method of constructing a macro pixel MP according to one or more exemplary embodiments.

Referring to FIG. 6A, when the biometric signal measurement apparatus 1 performs a binning operation for two adjacent lines for each of a plurality of row lines and column lines in an 8×8 pixel array 100, 16 macro pixels MP each having a size of 2×2 may be constructed. In this case, the total readout time may be reduced by ½, and the total number of analog-to-digital converters required to convert the pixel signal to a digital signal may be reduced by ½.

Referring to FIG. 6B, the living body or biometric signal measuring device 1 performs a binning operation on the first through m-th row lines ROW[1]-ROW[m], thereby generating n macro pixels MP each having m unit pixels PX for each of the column lines COL[1]-COL[n]. The binning operation for the row lines ROW[1]-ROW[m] may be performed by synchronizing the row line select signals. For example, when the first to m-th row lines ROW[1]-ROW[m] selection signals are simultaneously activated, the unit connected to the first to the m-th row lines ROW is formed. The pixels PX may be grouped by column lines COL[1]-COL[n].

Referring to FIG. 6C, the biometric signal measuring device 1 includes m row lines ROW[1]-ROW[m] and n column lines COL[1]-COL[n]. By performing a binning operation on a first row line ROW[1], a second row line ROW[2], a first column line COL[1], and a second column line COL[2], one macro pixel MP including four unit pixels PX may be constructed.

The binning operation for the first row line RO[1] and the second row line ROW[2] is performed by simultaneously applying the first row line (ROW[1]) select signal and the second row line (ROW[2]) select signal.

The binning operation for the column lines COL[1]-COL[n] is performed by the switching operation of the first through the n-th switches SW1-SWn arranged between the column lines COL[1]-COL[n]. For example, when the first switch SW1 between the first column line COL[1] and the second column line COL[2] is turned on, the first column line COL[1] and the second column line COL[2] may be grouped by each row line ROW[1]-ROW[m].

The biometric signal measuring device 1 may configure the macro pixels MP in various shapes and sizes based on the type of biometric signals, the dynamic range of the image sensor, and the like. For example, the biometric signal measuring device 1 may use a 14-bit dynamic range image sensor to measure 16-bit or more dynamic range suitable for measurement of the pulse wave signal PPG and sampling over 100 samples per sec (SPS). To achieve this rate, a macro pixel (MP) in the form of a square having a size of 4×4 may be constructed as shown in FIG. 7A. Referring to FIG. 7A, a living body or biometric signal measuring device 1 respectively bins four row lines and column lines adjacent to each other in an 8×8 pixel array 100 to form a total of four (4×4) macro pixels MP. In this case, the overall readout time may be reduced by ¼, and the total number of analog-to-digital converters required to convert the pixel signal to a digital signal may be reduced by ¼.

In addition, the biometric signal measuring device 1 may configure the macro pixels MP in various shapes and sizes based on the distance from the center of the pixel array 100. For example, in order to uniformly maintain the light sensitivity of the unit pixels PX, the biometric signal measuring device 1 may be configured to have a relatively large distance from the center of the pixel array 100. As shown in FIG. 7B, macro pixels MP having various sizes may be constructed. Referring to FIG. 7B, the pixel array 100 may include first through seventh macro pixels MP1-MP7. The first macro pixel MP1 located at the center C of the pixel array 100 may have a size of 2×2. The second macro pixel MP2 and the third macro pixel MP3 arranged at the first position spaced apart by d1 from the center C of the pixel array 100 have a size of 3×2, which is larger than the first macro pixel MP1. Further, the fourth to seventh macro pixels MP4 to MP7 arranged at the second position spaced apart by d2 (d2 is larger than d1) from the center C of the pixel array may have a size of 4×3, which is larger than the second macro pixel MP2 and the third macro pixel MP3. The biometric signal measuring device 1 constitutes macro pixels MP1-MP7 having a relatively large size as distance from the center of the pixel array 100 increases, thereby preventing a decrease in photosensitivity and improving the measurement accuracy of a living body signal.

Referring to FIG. 2, the controller 300 may include a plurality of circuits for controlling the pixel array 100. For example, in an exemplary embodiment, the controller 300 may include a row driver 310, a readout circuit 320, a control logic 330, and a light source driver 340.

The row driver 310 may drive the pixel array 100 on a row-by-row basis. For example, the row driver 310 may generate a transfer control signal for controlling the transfer transistor of the unit pixel PX, a reset control signal for controlling the reset transistor, a selection control signal for controlling the selection transistor, and the like on a row-by-row basis. In an exemplary embodiment, the row driver 310 may drive at least some of the plurality of row lines under the control of the binning controller 200 in synchronization. For example, when the first row line and the second row line are binned, the row driver 310 may simultaneously drive the first row line and the second row line. In this case, the unit pixels PX connected to the respective column lines are grouped to constitute the macro pixels MP, and the pixel signals of the grouped unit pixels PX are summed and output.

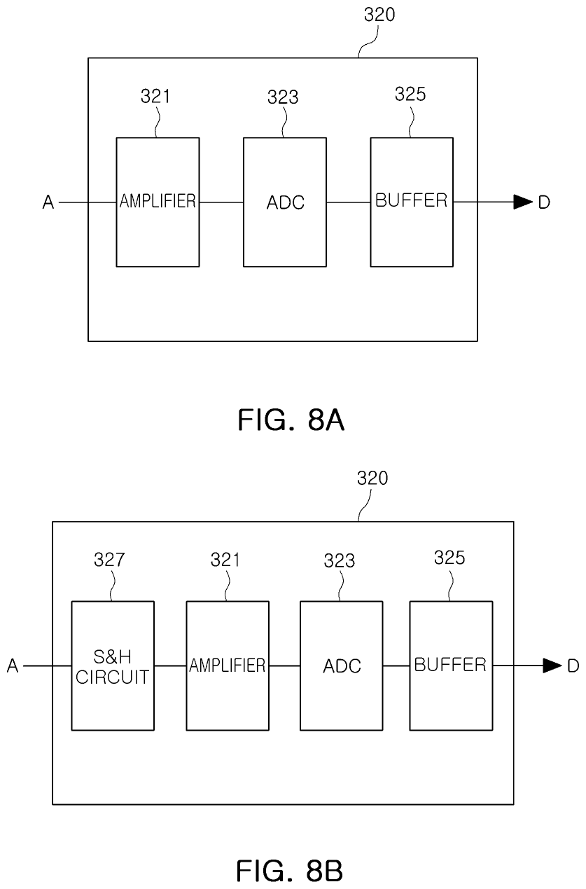

The readout circuit 320 may detect a pixel signal output from the unit pixels PX included in the pixel array 100 and convert the pixel signal into a digital signal. Examples of the readout circuit 320 according to various exemplary embodiments are shown in FIGS. 8A and 8B.

Referring to FIG. 8A, the readout circuit 320 may include an amplifier 321, an analog-to-digital converter 323, and a buffer 325.

The amplifier 321 may amplify the pixel signals output from the respective macro pixels MP with a predetermined amplification ratio.

In an exemplary embodiment, the amplification ratio of the amplifier 321 may be changed dynamically based on the current light amount of the light source 10. For example, when the current light amount of the light source 10 is equal to or greater than a predetermined threshold value, the control logic 330 may set the amplification ratio of the amplifier 321 to a first value. Conversely, when the current amount of light in the light source 10 is below a predetermined threshold, the control logic 330 may set the amplification ratio of the amplifier 321 to a second value greater than the first value.

In an exemplary embodiment, the amplifier 321 may analog-sum-amplify the pixel signals output from the macro pixels MP. For example, the amplifier 321 may sum the pixel signals output from the macro pixels MP, and amplify the summed pixel signals at a predetermined amplification ratio.

The analog-to-digital converter 323 may convert the pixel signal amplified by the amplifier 321 into a digital signal. The pixel signal converted into the digital signal may be stored in the buffer 325.

Referring to FIG. 8B, the readout circuit 320 may further include an S&H circuit 327. The S&H circuit 327 may store pixel signals output from the macro pixels MP. In an exemplary embodiment, the S&H circuit 327 may be provided for each macro pixel MP to separately store pixel signals.

Referring back to FIG. 2, the control logic 330 may control the row driver 310, the readout circuit 320, and the light source driver 340. The control logic 330 may include a timing generator 331, a digital binning unit 332, and the like.

The timing generator 331 may generate various timing signals for controlling the operation of the controller 300. For example, the timing generator 331 may generate a timing signal for synchronizing the driving timing of the light source 10 with the detection timing of the pixel signal, and output the timing signal to the light source driver 340. The biometric signal measurement apparatus 1 may minimize the driving time and power consumption of the light source 10 by synchronizing the driving timing of the light source 10 with the detection timing of the pixel signal.

The digital binning unit 332 may generate a biometric signal using the digital signals stored in the buffer 325. For example, the digital binning unit 332 may generate a living body (or biometric) signal by cumulatively averaging the digital values of the digital signals stored in the buffer 325.

The biometric signal generated by the digital binning unit 332 may be transmitted to an external processor or the like and be used to acquire biometric information of the object. For example, the external processor may analyze biometric signals transmitted from the digital binning unit 332 to obtain biological information such as blood pressure, blood vessel age, arteriosclerosis, aortic pressure waveform, and stress index of the object.

The light source driver 340 may generate a predetermined pulse signal to drive the light source 10. For example, the light source driver 340 may determine the period, the duty ratio, and the duration of the pulse signal based on the timing signal generated by the timing generator 331. In an exemplary embodiment, the light source driver 340 may drive the light source 10 in synchronization with the detection time of the pixel signal.

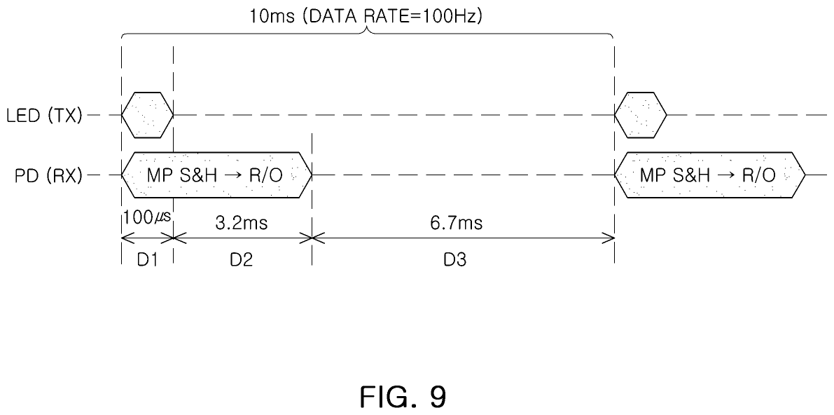

FIGS. 9 to 11 are views for explaining the operation of the biometric signal measuring device 1 according to an exemplary embodiment.

Referring to FIG. 9, a biometric signal measurement period (T=10 ms, data rate=100 Hz) includes a first section D1 for detecting a pixel signal from unit pixels by driving a light source, a second section D2 for converting into a digital signal, and a third section D3 operating in a power-down mode.

In the first section D1, the biometric signal measuring device 1 may drive a light source (e.g., LED) 10 and output a light signal toward a part of the user's body. The biometric signal measuring device 1 according to one or more exemplary embodiments controls the first section D1 to a very short time (e.g., 100 μs) in comparison with the second section D2 and the third section D3, thereby minimizing driving of the light source 10.

A photodiode (PD) of a biometric signal measuring device 1 may receive a light signal by a rolling shutter method or a global shutter method to generate charges. The biometric signal measuring device 1 may convert the charge generated by the photodiode PD into a pixel signal, and then add pixel signals by a predetermined pixel unit through an analog binning operation. In an exemplary embodiment, the biometric signal measuring device 1 may perform an analog binning operation by grouping unit pixels PX and constructing at least one macro pixel (MP). In this case, the biometric signal measuring device 1 may perform the readout operation for each macro pixel (MP), thereby reducing the readout time.

In the first section D1, the biometric signal measurement device 1 may perform a first operation for detecting pixel signals of the respective macro pixels MP. In an exemplary embodiment, the biometric signal measuring device 1 may store the detected pixel signal in the S&H circuit 327 provided for each macro pixel (MP).

In the second section D2, the biometric signal measurement device 1 may perform a second operation of converting the pixel signal detected from each macro pixel MP into a digital signal.

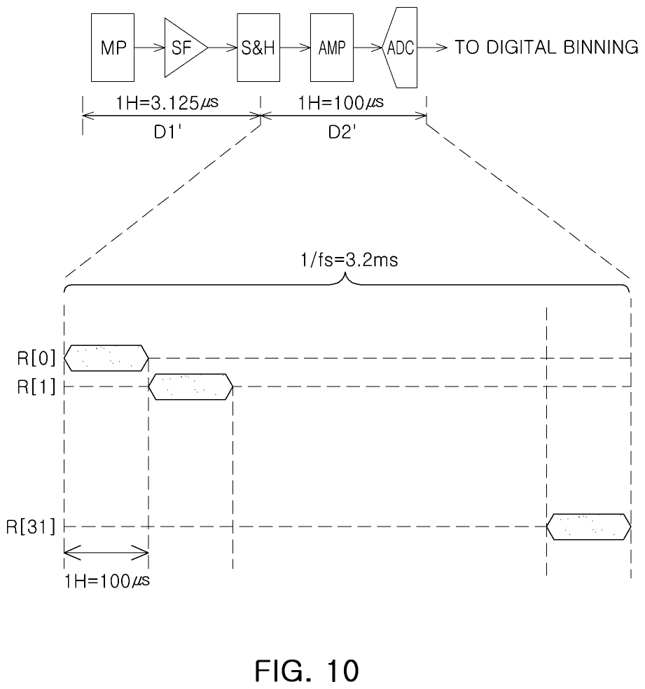

In an exemplary embodiment, the biometric signal measurement device 1 may sequentially perform the second operation on the pixel signal detected from each macro pixel (MP). Referring to FIG. 10, when unit pixels PX are grouped by each row line (R[0]-R[31]) to form a total of 32 macro pixels MP, The time for detecting the pixel signal from each macro pixel MP during the first section D1 may be 3.125 μs(=100 μs/32). In addition, the time to convert each pixel signal into a digital signal in the second section D2 may be 100 μs(=3.2 ms/32).

In an exemplary embodiment, the biometric signal measurement device 1 performs an analog summing amplification operation on all or a part of the pixel signals detected from the macro pixels MP, and the second operation may be performed simultaneously. For example, in the above example, when performing an analog summing amplification operation on pixel signals of all macro pixels, the second section D2 may be 100 μs. As a result, the manufacturing cost of the biometric signal measuring device 1 may be reduced by minimizing the total number of analog-to-digital converters (ADCs) required to convert the pixel signals into digital signals.

In the third section D3, the biometric signal measuring device 1 may perform a third operation of calculating the biometric signal using the pixel signals converted into the digital signal. In an exemplary embodiment, the biometric signal measurement device 1 may minimize power consumption by operating in a power-down mode that deactivates the functions of various components related to the light-emitting operation of the light source 10 and the light-receiving operation of the photodiode PD. In the example of FIG. 9, the third section D3 may be 6.7 ms and may be longer than the sum of the first section D1 and the second section D2.

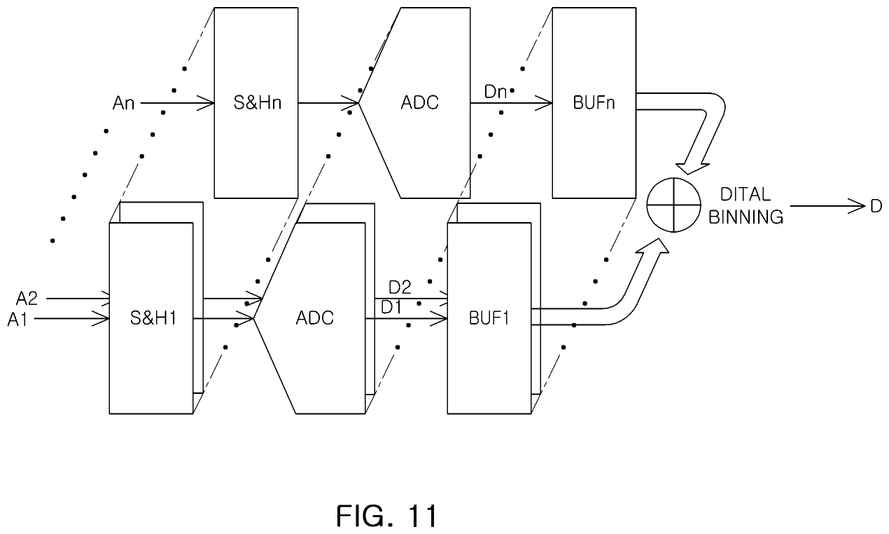

The first to third operations of the biometric signal measuring device are summarized as shown in FIG. 11.

Referring to FIG. 11, the pixel signals A1-An output from the macro pixels MP may be stored in the S&H circuits S&h1 and S&Hn (first operation). The S&H circuit may be provided separately for each macro pixel (MP). The pixel signals A1-An may be converted to digital signals D1-Dn via an analog-to-digital converter (ADC) (second operation). The analog-to-digital converter (ADC) may be provided separately for each macro pixel (MP) or may be provided in common to one or more macro pixels (MP). The digital signals D1-Dn may be stored in buffers BUF1-BUFn.

Thereafter, the biometric signal measurement device 1 may generate the biometric signal D by performing a digital binning operation on the digital signals D1-Dn stored in the buffers BUF1-BUFn. For example, the biometric signal measuring device 1 may generate the biometric signal D by cumulatively averaging the digital values of the digital signals D1-Dn.

In an exemplary embodiment, the biometric signal measurement device 1 may perform a digital binning operation after applying predetermined weights to the digital values of each of the digital signals D1-Dn. For example, the biometric signal measurement device 1 applies a first weight to the first macro pixel MP, and a second weight, greater than the first weight, to the second macro pixel MP2, which has a larger distance from the center of the pixel array than the first macro pixel MP1, after which a digital binning operation may be performed. The biometric signal measurement apparatus may improve the measurement accuracy by applying different weights to each of the digital signals (D1-Dn) based on the distance from the center of the pixel array and the like.

The biometric signal D generated from the pixel signals A1-An may be transmitted to a processor (e.g., external processor) and be used to acquire biometric information of the user.

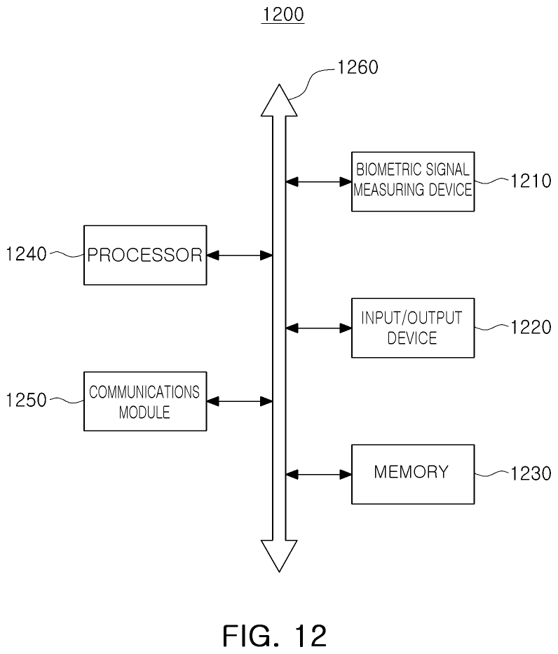

FIG. 12 is a block diagram of an electronic device 1200 including a biometric signal measurement device 1210 according to an exemplary embodiment.

Referring to FIG. 12, the electronic device 1200 may include a biometric signal measurement device 1210, an input/output device 1220, a memory 1230, a processor 1240, and a communication module 1250. The electronic device 1200 may be a smart phone, a tablet PC, a smart wearable device, a mobile device, a smart wearable device, etc.

The biometric signal measurement device 1210 is as described above with reference to FIGS. 1 to 3, 4A, 4B, 5A to 5C, 6A to 6C, 7A, 7B, 8A, 8B, and 9 to 11, and may be mounted on a package substrate or the like and connected to the processor 1240 through a bus 1260 or other communication means.

The input/output device 1220 may include an input device such as a keyboard, a mouse, a touch screen, etc., and an output device, such as a display, an audio output, etc.

The memory 1230 may be a storage medium for storing data used in the operation of the electronic device 1200, or multimedia data. Memory 1230 may include volatile memory, or non-volatile memory such as flash memory and the like. Also, the memory 1230 may include at least one of a solid state drive (SSD), a hard disk drive (HDD), and an optical drive (ODD) as a storage device.

A processor 1240 (e.g., at least one processor) may perform particular operations, commands, tasks, and so on. The processor 1240 may be a central processing unit (CPU) or microprocessor unit (MCU), a system on chip (SoC), etc., and may be coupled to the biometric signal measurement device 1210, the display 1220, 1230, as well as other devices connected through the communication module 1250 (e.g., communicator, communication interface, etc.).

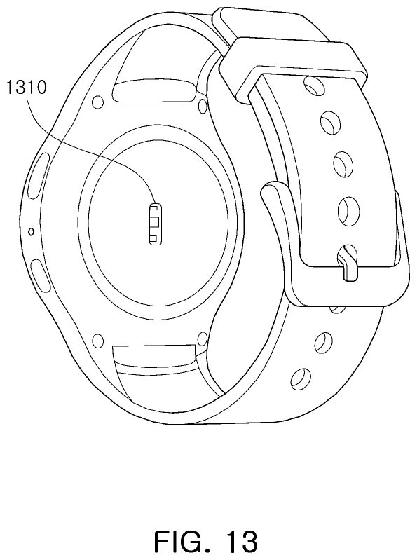

FIGS. 13 to 15 are views illustrating examples of an electronic device 1300, 1400, and 1500 including a biometric signal measurement apparatus or device 1310, 1410, and 1510 according to one or more exemplary embodiments.

First, referring to FIG. 13, the electronic device 1300 may be implemented as a watch type wearable device.

The electronic device 1300 may include a body and a wrist strap. A display is provided on the front of the main body, and various application screens including time information, received message information, and the like may be displayed. The user may wear the electronic device 1300 on the wrist using the strap.

A biometric signal measuring device 1310 may be disposed on the rear surface of the main body. The living body or biometric signal measuring device 1310 may output a light signal to a body region that is in contact with the back surface of the main body such as a wrist of a user, and may measure the living body or biometric signal by sensing the reflected light reflected from the body region. The electronic device 1300 may analyze the biometric signal measured by the biometric signal measuring device 1310 to acquire biometric information of the user such as blood pressure, blood vessel age, arteriosclerosis, aortic pressure waveform, a stress index, or the like.

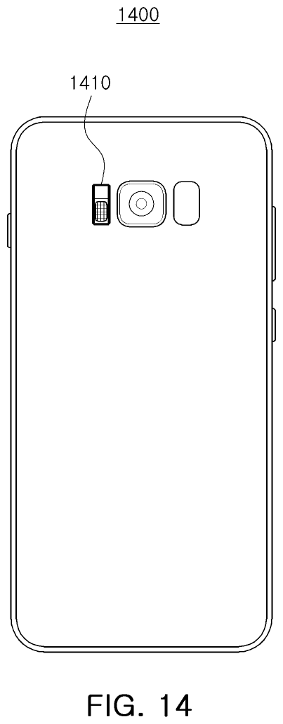

Referring to FIG. 14, the electronic device 1400 may be implemented as a mobile device such as a smartphone.

The electronic device 1400 may include a housing and a display panel.

The housing may form the appearance of the electronic device 1400. The housing may include a first surface, a second surface opposite the first surface, and a side surface surrounding the space between the first surface and the second surface.

A display panel and a cover glass may be sequentially arranged on the first surface of the housing. The display panel may be exposed to the outside through the cover glass.

On the second side of the housing, a biometric signal measuring device 1410, a camera module and an infrared sensor may be disposed.

When a user requests biometric information by executing an application or the like mounted on the electronic device 1400, the biometric signal measuring device 1410 may measure the biometric signal by sensing reflected light obtained from a part of the user's body. The electronic device 1400 may acquire biometric information of the user by analyzing the biometric signal measured by the biometric signal measuring device 1410.

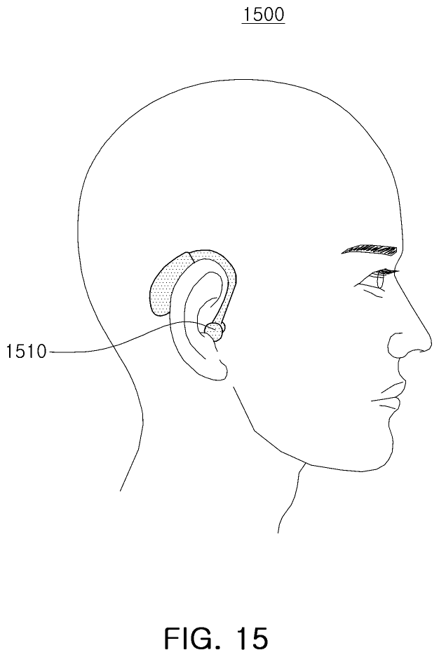

Referring to FIG. 15, the electronic device 1500 may also be implemented as an ear wearable device.

The electronic device 1500 may include a body and an ear strap.

The user may wear the electronic device 1500 by wearing the ear strap on the auricle. With the user wearing the electronic device 1500, the main body may be inserted into the external auditory meatus of the user.

The body may be equipped with a biometric signal measuring device 1510. The living body or biometric signal measuring device 1510 may output a light signal to a body region that is in contact with the body, such as a wall surface of a user's ear canal, and may measure a living body signal by sensing reflected light reflected from the body region. The wall of the external ear canal of the user is thinner than other areas of the body, so that it is easy to measure the biometric signals such as blood flow. The electronic device 1500 may acquire biometric information of the user by analyzing the biometric signal measured by the biometric signal measuring device 1510.

As set forth above, an apparatus for measuring biometric signals according to one or more exemplary embodiments may minimize size and power consumption by using an image sensor.

In addition, the biometric signal measuring device according to one or more exemplary embodiments minimizes the number of analog-digital converters required to generate a biometric signal, thereby minimizing the manufacturing cost.

Furthermore, the biometric signal measuring device according to one or more exemplary embodiments may minimize the size and noise by implementing the light receiving unit and the driving circuit on a single chip.

The various advantages and effects of the present inventive concept are not limited to the above description.

While exemplary embodiments have been shown and described above, it will be apparent to those skilled in the art that modifications and variations could be made without departing from the scope of the present inventive concept as defined by at least the appended claims.

1. A biometric signal measuring device comprising:
a light source configured to output a light signal to an object;
a pixel array connected to a plurality of row lines and a plurality of column lines, and comprising a plurality of unit pixels configured to sense a reflected light signal corresponding to a reflection of the output light signal by the object;
a binning controller configured to group the plurality of unit pixels to obtain one or more macro pixels; and
a controller configured to drive the light source and to obtain a biometric signal of the object, based on a pixel signal generated by each of the one or more macro pixels according to the reflected light signal.

2. The biometric signal measuring device of claim 1, wherein the one or more macro pixels comprise a first macro pixel including a first number of unit pixels, among the plurality of unit pixels, and a second macro pixel including a second number of unit pixels, among the plurality of unit pixels, the second number being greater than the first number.

3. The biometric signal measuring device of claim 2, wherein the first macro pixel is disposed in a first location of the pixel array, and the second macro pixel is disposed in a second location of the pixel array, the second location being farther from a center of the pixel array than the first location.

4. The biometric signal measuring device of claim 1, wherein the controller comprises:
an analog-to-digital converter configured to convert the pixel signal into a digital signal; and
a biometric signal generator configured to obtain the biometric signal by applying a predetermined weight to the digital signal and summing the predetermined weight and the digital signal.

5. The biometric signal measuring device of claim 4, wherein:
the one or more macro pixels comprise a first macro pixel disposed in a first location of the pixel array, and a second macro pixel disposed in a second location of the pixel array, the second location being farther from a center of the pixel array than the first location; and
a first weight value applied to a first digital signal converted from a first pixel signal generated by the first macro pixel is greater than a second weight value applied to a second digital signal converted from a second pixel signal generated by the second macro pixel.

6. The biometric signal measuring device of claim 4, wherein the controller further comprises a light source driver configured to synchronize a driving timing of the light source with a detection timing of the pixel signal.

7. The biometric signal measuring device of claim 4, wherein:
the controller further comprises an amplifier configured to amplify the pixel signal; and
the analog-to-digital converter is configured to convert the pixel signal amplified by the amplifier into the digital signal.

8. The biometric signal measuring device of claim 1, wherein a first time interval in which the controller detects and stores the pixel signal is shorter than a second time interval in which the controller converts the stored pixel signal into a digital signal.

9. The biometric signal measuring device of claim 1, wherein the controller is configured to convert the pixel signal into a digital signal in a state in which the light source is turned off.

10. The biometric signal measuring device of claim 1, further comprising an optical filter and a microlens disposed on an upper portion of the light source.

11. The biometric signal measuring device of claim 10, wherein the optical filter comprises a near-infrared filter through which only a light signal in a near-infrared region in the reflected light signal passes.

12. A biometric signal measuring device comprising:
a pixel array comprising a plurality of unit pixels, the plurality of unit pixels being grouped into one or more macro pixels;
a readout circuit configured to detect, during a first time period, a pixel signal output by each of the one or more macro pixels based on an external light signal, and to convert the detected pixel signal into a digital signal during a second time period; and
a control logic configured to control operation timings of the pixel array and the readout circuit, and to sum the digital signal to generate a biometric signal.

13. The biometric signal measuring device of claim 12, wherein during the first time period, a photodiode included in each of the unit pixels is configured to generate charge based on the external light signal.

14. The biometric signal measuring device of claim 12, further comprising a light source configured to output a light signal to an object,
wherein the pixel array is configured to sense the external light signal as a reflection of the output light signal reflected from the object.

15. The biometric signal measuring device of claim 14, wherein the light source is configured to output the light signal to the object in a global shutter manner.

16. The biometric signal measuring device of claim 12, wherein the first time period is shorter than the second time period.

17. The biometric signal measuring device of claim 12, wherein the readout circuit is configured to sequentially scan row lines connected to the one or more macro pixels to detect the pixel signal from each of the one or more macro pixels.

18. A biometric signal measuring device comprising:
a plurality of unit pixels connected to a plurality of row lines and a plurality of column lines;
a binning controller configured to group the plurality of unit pixels to obtain one or more macro pixels; and
a controller configured to convert a pixel signal generated by each of the one or more macro pixels based on an external light signal into a digital signal, and to generate a biometric signal by applying a predetermined weight to the digital signal.

19. The biometric signal measuring device of claim 18, wherein the binning controller is configured to generate the one or more macro pixels by binning the plurality of row lines.

20. The biometric signal measuring device of claim 19, wherein the pixel signal is generated by summing output signals of unit pixels included in each of the one or more macro pixels.

21-25. (canceled)
